(19) United States
(12) Patent Application Publication
(10) Pub. No.: US 2005/0037041 A1
(43) Pub. Date: Feb. 17, 2005

(54) Duplex coated color lake and other powders, their preparation and cosmetic and other uses

(76) Inventors: David Schlossman, Short Hills, NJ (US); Yun Shao, Piscataway, NJ (US); Frank Mazzella, Rhineback, NY (US); Charles Quinn, Purdys, NY (US)

Correspondence Address: ANTHONY H. HANDAL;KIRKPATRICK & LOCKHART, LLP, 599 LEXINGTON AVENUE, 31ST FLOOR, NEW YORK, NY, 10022-6030, US

(21) Appl. No.: 10/856,336

(22) Filed: May 28, 2004

Related U.S. Application Data
(63) Continuation-in-part of application No. 10/791,424, filed on Mar. 1, 2004.
(60) Provisional application No. 60/451,056, filed on Feb. 28, 2003.

(30) Foreign Application Priority Data
Mar. 2, 2004 (WO) PCT/US04/06300

(51) Int. Cl. A61K 7/021; A61K 7/00

(52) U.S. Cl. 424/401; 424/63; 264/4

(57) ABSTRACT

Hard-to-coat color lakes and other powders useful in cosmetics and other industries are coated with a duplex coating system comprising basic and nonbasic functionalized coating components, for example a basic functionalized aminopolysiloxane and a nonbasic alkoxypolysiloxane. Control of bleeding of a color lake into aqueous media is possible. A novel duplex coating system, treatment process, treated powder and cosmetic formulation containing same are disclosed.

### CROSS-REFERENCE TO RELATED APPLICATIONS

This application is a continuation-in-part of David Schlossman et al., copending U.S. patent application Ser. No. 10/791,424 filed Mar. 1, 2004, attorney docket no. DS420 and of corresponding commonly owned international PCT Patent Application No. PCT/US2004/006300 filed Mar. 2, 2004, attorney docket no. DS420PCT. The disclosures of the aforementioned United States and international patent applications are hereby incorporated herein by reference thereto.

### STATEMENT REGARDING FEDERALLY SPONSORED RESEARCH OR DEVELOPMENT

(Not applicable.)

### BACKGROUND OF THE INVENTION

The present invention relates to novel duplex coated color lake and other powders, their preparation and uses. The invention includes methods of treatment color lake powders, and other powders, to the products of the treatments, and to end-product formulations and methods incorporating the treated powders. The novel treated powders of the invention are hydrophobic, or water-repellent, and have particular, but not exclusive, application in the cosmetics industry. They may also have uses in other industries, for example, the foodstuffs, paint and plastics industries, as will be apparent to those skilled in the art.

Insoluble powder materials, for example colorful pigments, sunscreen agents, talc and the like, are commonly employed in the cosmetics and other industries, such as the paint, coatings and plastics industries, to serve a variety of purposes. Suitable powders may impart qualities of color, opacity or special visual effects, such as pearlescence, or other qualities such as bulk, feel and oil absorbency, to a wide range of consumer and industrial products. Such powders are generally insoluble in either aqueous or organic media.

For good end-product quality and homogeneity it is desirable for the insoluble powders to be uniformly dispersed in suitable media, which may be other powders, or aqueous, lipid or silicone media. Poor dispersion may lead to aggregation or agglomeration into larger particles with non-uniform end-product appearance, streaking, settling, poor feel or other product drawbacks. Good dispersibility is generally thought to be obtainable by completely coating the outer surfaces of the powder particles with a hydrophobic material, for example an organosilicon material. However, difficulties may arise if the coating is not complete or does not endure. Defects in the coating may expose hydrophilic material leading to particle agglomeration.

Some powders are particularly difficult to coat owing to their poor surface reactivity or for other reasons. For example, micas such as sericites or other pigment materials employed in cosmetics and other products for the lustrous appearance they impart are notably unreactive and hard to coat. Furthermore, color lakes which typically comprise a dye absorbed on an insoluble substrate such as alumina are prone to bleed the dyestuff into aqueous media. Known hydrophobic coatings do not effectively prevent such bleeding.

Bellanca, et al. U.S. Pat. No. 4,167,422 describes problems of bleeding and other drawbacks of color lakes including FD&C (U.S. food, drug and cosmetic) certified color lakes.

Silicone compounds are noted for their hydrophobicity and have therefore been used as coating materials for cosmetic and other powders. Known hydrophobic treatments for cosmetic powders, notably inorganic and organic pigments and fillers, include a number of organosilicon compounds, for example dimethylpolysiloxanes having a backbone of repeating —Me₂SiO— units (“Me” is methyl, CH3), methyl hydrogen polysiloxanes having a backbone of repeating —MeHSiO— units and alkoxysilanes of formula Rₙ—SiH₍₄₋ₙ₎ where “R” is alkyl and “n” is the integer 1, 2 or 3. The resultant organosilicon-treated pigments or fillers are considered useful in cosmetic products such as long-lasting liquid makeup and other two-phase, oil-in-water or water-in-oil cosmetics.

Witucki in “A silane primer: Chemistry and Applications of Alkoxy Silanes” Journal of Coatings Technology 65;822 pages 57-60 duly 1993) discusses use of alkoxy functional silanes for surface treatment of inter alia particulate pigments and fillers. Described reaction mechanisms include hydrogen bonding to surface hydroxy groups followed by drying or curing with elimination of water to form a covalent bond from each alkoxy-bearing silicon atom to the particle substrate.

In this vein, Hollenberg et al. U.S. Pat. No. 5,143,722 describes the coating of cosmetic pigments with hydrophobic materials comprising dimethylpolysiloxane materials, including cross-linked products. The coatings are prepared from liquid polymerizable silicone starting materials having reactive terminal groups such as hydroxyl or alkoxy groups, by heating slurries of the pigment particles mixed with the starting materials.

Published Japanese patent application JPA 7-196946 (Miyoshi Kasei KK) discloses the use of a straight chain alkylpolysiloxanes having reactive terminal groups such as alkoxy, hydroxy, halogen, amino or imino groups for treating pigments. A similar approach for coating cosmetic powders is disclosed in Hasegawa U.S. Pat. No. 5,458,681, where alkylpolysiloxanes with a specific narrow distribution of molecular weight are employed, namely a ratio of weight-average molecular weight to number average molecular weight of from 1.0 to 1.3.

Some additional organosilicon compounds that have been descried as starting materials in the surface treatment of cosmetic powders are alkoxysilanes for example alkyltriethoxy or alkyltrimethoxysilanes such as SILQUEST (trademark) A-137 silane available from OSI Specialities or PROSIL 9202 available from PCR. According to the manufacturer, SILQUEST A-137 silane is a monomeric alkyl alkoxysilane that when exposed to moisture is reactive with the minerals in concrete, masonry and other substrates to penetrate and protectively coat the substrate particles.

Glausch et al. U.S. Pat. No. 6,176,918 discloses a method of coating mica-based modified pearl luster pigments employing an oligomeric silane system. However Glausch et al.'s coating is intended to provide hydrophilicity or water-compatibility, not hydrophobicity or water repellency and accordingly is not relevant to the objectives of the present invention.

Though not known to the art prior to the present invention, commonly owned U.S. patent application Ser. No. 10/293,745 filed Nov. 13, 2002 (attorney docket number DS310) and corresponding international patent publication number WO 03/043567 in the name of assignee Kobo Products, Inc., published 30 May 2003 (attorney docket number DS310PCT), not more than one year prior to the date of filing of the present application, disclose the use of an organosilicon coating agent containing basic groups such as amino groups to coat hard-to-treat cosmetic pigments such as sericites. The organosilicon coating agent may comprise a blend of organosilicon compounds one of which has basic groups and the other of which does not. The aforesaid U.S. and international patent applications are referenced herein as “the DS310 applications”. The entire disclosure of patent application Ser. No. 10/293,745 is hereby incorporated herein by this specific reference thereto. The invention disclosed in the DS310 applications, neither addresses the problems addressed herein nor provides a solution to same.

The foregoing description of background art may include insights, discoveries, understandings or disclosures, or associations together of disclosures, that were not known to the relevant art prior to the present invention but which were provided by the invention. Some such contributions of the invention may have been specifically pointed out herein, whereas other such contributions of the invention will be apparent from their context. Merely because a document may have been cited here, no admission is made that the field of the document, which may be quite different from that of the invention, is analogous to the field or fields of the present invention.

### BRIEF SUMMARY OF THE INVENTION

Having regard to the foregoing drawbacks of the art, there is a need for a hydrophobic treatment process that is effective in treating hard-to-coat cosmetic pigments and other insoluble powders. Furthermore, there is a need for a powder coating treatment which can control bleeding of color lakes into aqueous media. An additional desirable objective would be to provide a treatment for insoluble powders and treated powders that overcome one or more of the foregoing drawbacks and are also applicable to a wide range of cosmetic powder materials.

To solve the aforesaid problem, and to fulfill the above and other objects, the invention provides a process for treating a powder, optionally a cosmetic powder, to render the powder hydrophobic, the process comprising treating the powder with a duplex coating system comprising an effective amount of a basic coating component and an effective amount of a nonbasic coating component, by applying the basic coating component to the powder so as to substantially coat the powder with the basic coating component, then applying the nonbasic coating component with mixing and curing the resultant powder coated with the basic and nonbasic coating components.

Usefully, the basic and the nonbasic components of the duplex coating system can each comprises a functionalized organosilicon compound, the basic component functionalized organosilicon compound containing reactive basic groups and, optionally, a second organosilicon compound lacking reactive basic groups.

In one embodiment, the nonbasic coating component comprises a functionalized silane having from one to four silicon atoms and the basic coating component comprises a polysiloxane substituted with multiple amino or other basic nitrogen groups and with multiple methoxy or ethoxy groups.

Some useful powders to be treated are cosmetic powders, color lakes, organic pigment powders, hard-to-coat powders, powders that have hydrophilic outer surfaces and mixtures of the foregoing powders.

With advantage, the nonbasic coating component is applied to the powder after application of the basic coating component has been completed, in a two-stage treatment.

The invention includes the coated powders produced by the above-described method as well as cosmetic compositions comprising from about 0.1 to about 99 percent by weight of such coated powder.

Color lakes, for example Yellow No. 5 Alumina Lake, coated by the process of the invention may exhibit good hydrophobicity and reduced bleed in aqueous media.

### DETAILED DESCRIPTION OF THE INVENTION

Some embodiments of the invention, and of making and using the invention, as well as the best mode contemplated of carrying out the invention, are described in detail below. The following more detailed description of the invention is intended to be read in the light of, or in context with, the preceding summary and background descriptions which may include pertinent description of the invention as will be apparent to those skilled in the art.

In one broad aspect the invention provides a novel treatment for color lakes and other powders, employing a novel duplex coating system, which treatment is effective to render the coated powders hydrophobic. The novel treatment may be useful to control bleeding of colorant or other undesired agent from the powder into liquid media, especially aqueous liquid media. The powder or powders to be treated may include, without limitation, organic pigment powders and other powders, especially cosmetic powders, powders that are hard to coat and powders that have notably hydrophilic outer surfaces.

One useful embodiment of the duplex coating system for use in this treatment process comprises respective basic and nonbasic coating components each of which is or comprises a functionalized organosilicon compound or compounds. A suitable basic coating component comprises a functionalized organosilicon compound containing reactive basic groups and may optionally comprise a second organosilicon compound which lacks reactive basic groups. A useful embodiment of nonbasic coating component comprises a functionalized silane or other functionalized nonbasic organosilicon coating compound that yields a hydrophobic cross-linked reaction product upon curing. Desirably the reaction product is essentially nonpolar and can be formed as a coherent layer on powder substrates to which the first basic organosilicon component has been applied. In each case the functionalization of the organosilicon compound can comprise one or preferably more alkoxy groups, as is described in more detail hereinbelow.

By way of nonlimiting example, the basic compound can comprise a polysiloxane substituted with multiple amino or other basic nitrogen groups and with multiple methoxy or ethoxy groups. An example of a suitable nonbasic functionalized silane is a trimethoxy- or triethoxy-alkylsilane having a single silicon atom. Dialkoxy analogs or homologs thereof are contemplated as being useful for the purposes of the invention but are not readily available commercially.

In one desirable embodiment of the invention the nonbasic component is applied to the powder after application of the basic component has been completed, providing a two-stage treatment.

Alternatively, the nonbasic coating component may be applied substantially after the basic component, meaning that there is some overlap whereby some nonbasic component is applied before application of the basic component is complete. For example the process may have three stages: a first stage wherein only the basic component is applied; a second stage wherein both first and nonbasic components are applied and a third stage wherein only the nonbasic component is applied. Preferably, the nonbasic component is not applied until all or substantially all powder surfaces have been contacted by the basic component.

Pursuant to a further alternative embodiment, the first and nonbasic components are applied simultaneously, optionally after having been blended together.

Desirably, the powder and coating components are thoroughly mixed to so that the coating components contact the outer surfaces of the particles as completely as is practicable. Ideally, the complete outer surfaces of all the particles may be contacted with both components. In one useful embodiment, the particles are contacted as completely as practicable by the first component and are then contacted as completely as practicable by the second component.

After the particles are coated with the duplex coating system, desirably they are subjected to curing. Curing can be effected as is known in the art, for example at an elevated temperature for a limited period of time.

Basic Functionalized Organosilicon Component. The basic functionalized organosilicon component can comprise a polymeric organosilicon compound having a backbone comprising a limited number of repeating siloxy units of hydrophobic character bearing basic groups, for example amino or amino-containing groups or other basic nitrogen groups. With advantage, the basic groups are preferably substituted in the repeating units of the backbone. The basic groups are believed to facilitate binding to certain desired substrates including color lakes which may have a distinctly hydrophilic surface character.

Optionally, the polymeric organosilicon compound may also bear anionic or electronegative reactive groups as substituents in the backbone siloxy units, or constituting terminal groups, or both. The reactive groups are preferably alkoxy groups but could be other suitable reactive groups such as hydroxyl, ether, keto, carboxyl, ethylene or chloro groups. The reactive groups may bind to the surface of the powder particles to be coated and may also, under the conditions of the coating reaction bind, to a limited extent, to other molecules of the organosilicon compound, polymerizing it.

The basic groups can be substituted in those repeating units that carry anionic reactive groups. In this case the so-functionalized organosilicon compound may be described as “amphoteric”, being a compound or unit having both acidic and basic characteristics.

The basic coating component of the duplex coating system can include, in addition to the basic organosilicon compound a supplemental organosilicon compound having similar structural characteristics to the basic organosilicon compound, including anionic reactive groups, for example methoxy or ethoxy groups, but lacking basic reactive groups. Some useful such organosilicon compounds lacking basic groups are also polymeric having one or more chains of siloxy units. Both the basic organosilicon compound and the further organosilicon compound are preferably liquids and the basic coating component can comprise a blend of the two liquids.

The amino or other basic group in the basic coating component has a good affinity for powders of interest in practicing the invention, and this affinity enhances the adsorption and the wetting of the basic coating component on the surfaces of substrate powder particles. The alkaline nature of the amino or other basic group also enables the group to catalyze the hydrolysis of alkoxy or other anionic reactive groups in either the basic organosilicon compound or the supplemental organosilicon compound which lacks basic groups.

A preferred basic group is an amine functional group. The basic groups can be the same or different in each molecule of the silicone compound and preferably comprise nitrogen-containing alkyl groups or a heterogenous nitrogen-carbon chain. The basic groups can comprise a primary amino group which can terminate an alkyl chain which can optionally also have one or more secondary amino groups in the chain in addition to the terminal primary amino group. Alternatively, the basic group can comprise a secondary or tertiary amine having lower alkyl substituents. A quaternary ammonium group, if employed as a basic substituent in the basic organosilicon compound is preferably present to a relatively low degree in view of the strongly cationic character of quaternary ammonium groups. The basic organosilicon compound is preferably also fully saturated and is terminated with alkoxy or alkyl groups.

Preferably the alkyl groups employed in the organosilicon compound are lower alkyl groups having no more than ten carbon atoms. More preferably, referring to nonbasic substituents in the organosilicon compound, the alkyl groups have no more than five carbon atoms and still more preferably are methyl or ethyl groups.

In some useful embodiments, the organosilicon compound is free of reactive groups other than those specified herein. For example, it is preferred that the basic organosilicon compound be free of hydroxyl, thio, carboxyl, chloro, nitro groups and unsaturation. Thus the basic organosilicon compound can consist essentially of dialkyl siloxy groups, alkoxy groups and basic groups. While the alkyl substituents in the siloxy groups can each be the same as one another, most preferably methyl groups or possibly ethyl or other groups, it will be understood that different alkyl groups, for example methyl, ethyl and butyl groups, may be present in the same molecule.

It is contemplated that the substitutions of alkoxy and basic groups in the dialkyl polysiloxane will be made directly into the silicon atoms of the backbone, but it is to be understood that substitution into one or both of the dialkyl groups may be possible, provided that the resultant organosilicon compound meets with the guidelines and objectives of the invention as described herein.

The proportion of siloxy groups without basic groups to siloxy groups bearing basic groups in the basic organosilicon compound can vary widely, for example from about 5:1 to about 1:5, but is preferably from about 2:1 to about 1:2, more preferably about 1:1.

The number of siloxy groups in the basic organosilicon compound can vary widely, for example from about 2 to about 200, but is preferably from about 5 to about 100, more preferably from about 5 to about 30 and still more preferably from about 10 to about 15.

The basic coating component can comprise a single homogenous basic organosilicon compound as described hereinabove, or can be a heterogenous mixture of two or more such basic organosilicon compounds having different structures meeting the criteria described herein.

One useful class of basic organosilicon compounds for use in the duplex coating system of the invention comprises compounds complying with the following Formula (1): 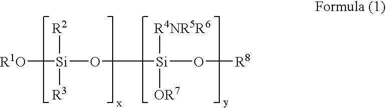
wherein
R¹, R⁷ and R⁸ are independently hydrogen or lower alkyl and are preferably methyl or ethyl;
R² and R³ are lower alkyl and are preferably methyl or ethyl;
R⁴ is a divalent lower alkyl group having formula —CₙH₂ₙ— where “n” is an integer from 1 to 10 preferably from 2 to 4 and more preferably is propylene;
R⁵ is hydrogen or lower alkyl and is preferably hydrogen;
R6 is hydrogen, lower alkyl or amino lower alkyl;
(x+y) is from 5 to 100, preferably from 10 to 15; and
x:y is from about 5:1 to about 1:5, preferably from about 2:1 to about 1:2 and is more preferably about 1:1, optionally about 1.2:1 to 1:1.2.

“Lower alkyl” is used herein to reference an alkyl group having from one to ten carbon atoms, preferably from 1 to 5 carbon atoms and more preferably methyl or ethyl. The value of (x+y) indicates the degree of polymerization and number of units in the polysiloxane.

Some useful embodiments of Formula 1 compounds comprise compounds wherein R¹, R², R³, R⁷ and R⁸ are methyl and R⁵ is hydrogen.

One useful embodiment of duplex coating system includes, as the basic coating agent, a blend of organosilicon compounds of the following formulas (2) and (3): 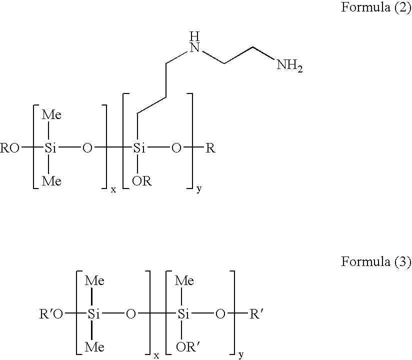
wherein Me is methyl;
R is methyl or ethyl;
R′ is methyl or ethyl; and

(x+y) is from 5 to 100, preferably from 10 to 15; and
x:y is from about 2:1 to about 1:2 and is more preferably about 1:1, optionally about 1.2:1 to 1:1.2.

The ratio of the compound of Formula (2) to the compound of Formula (3) in the blend can be any effective proportion, but is preferably in the range of from about 0.2:1 to about 5:1, and preferably about 1:1, for example from about 1.2:1 to about 1:1.2, on a weight basis. One preferred such blend is an amine functional silicone fluid available from GE Silicones, Waterford, N.Y., under the product code SF 1706. According to its data sheet, the product has a viscosity at 25° C. of 10-50 centistokes, a specific gravity at 25° C. of 0.986 and closed cup flash point of 95° C. and an amine equivalent of 0.48 milliequivalents of base/gram. Also, the product is said to have a 100 percent silicone content and to be soluble in most aromatic hydrocarbons.

A blend of compounds according to Formulas (2) and (3), for example the GE Silicones SF 1706 product, can be useful for hydrophobizing a variety of cosmetic powders. For example, a coating employing such a blended organosilicon coating agent can reduce the surface activity of titanium dioxide, zinc oxide and iron oxide, can facilitate the dispersion of particulate in oil, ester and silicone and can control the color shift that typically occurs with colored pigments when the pigments are wetted. Of particular interest to the present invention is the contribution of this basic component of the duplex coating system to the effective treatment of color lake powders so as, inter alia, to control bleeding into aqueous media.

Nonbasic Coating Component. Embodiments of the nonbasic component of the duplex coating system, as the name implies, lack basic groups. Some useful embodiments comprise a functionalized silane compound having a structure illustrated by the following Formula 4:
(R³O—)ₐ—Si—(—R⁹)_{b} (4)
wherein:
R³ is lower alkyl as defined hereinabove, optionally methyl, ethyl; propyl or butyl;
R⁹ is a saturated, unsaturated or polyunsaturated, straight chain, branched, unsubstituted cyclic, substituted cyclic alkyl or alkyl phenyl group having from 3 to 60 carbon atoms, preferably a saturated alky group having from 7 to 25 carbon atoms; and a+b=4.

The structures depicted in Formula (4) include mono- and dialkoxy silanes in addition to trialkoxysilanes, being compounds which can react with many powder surfaces. However, di- and tri-alkoxy silanes are advantageous for employment I in the present invention for their abilities to form polymers (or oligomers) and crosslinked networks which are chemically and physically stable. In practice, trialkoxysilanes, such as those specifically mentioned herein are particularly suitable for employment in the invention being suitably reactive and commercially available.

Some useful examples of the above-described functionalized silane compounds that can be used in or as the nonbasic coating component of the duplex coating system of the invention have the following Formula 5: 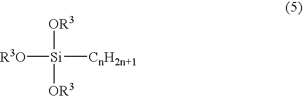
wherein R³ and n are the same as above and preferably R³ is methyl or ethyl and n is from 7 to 25. While not so limited, it will be appreciated that the functionalized silanes described or defined by Formulae (4) and (5) have a single silicon atom. Another useful class of functionalized silanes that can be employed in the present invention comprises analogous and homologous silanes to the Formulae (4) and (5) compounds and which have from 2 to 4 silicon atoms.

Some further examples of suitable functionalized silanes include: organoalkoxysilanes having an organic group or groups which may be unsubstituted or substituted or a mixture of different groups including for example, methyltrimethoxyalkylsilane, phenyltrimethoxyalkylsilane, and diphenyldimethoxy alkylsilane, as well as silanes having aryl-substituted organic groups, for example, gamma-methacryloxypropyl-trimethoxysilane wherein the alkyl group preferably has from 7 to 25 carbon atoms, more preferably from 8 to 12 carbon atoms and the aryl group is preferably a saturated hydrocarbon, save for benzene ring unsaturation, for example phenyl or alkylphenyl with up to 25 carbon atoms.

Other nonbasic functionalized silicon compounds that can be employed in or as the nonbasic coating component of the duplex coating system of the invention, including functionalized polysiloxane compounds, will be apparent to those skilled in the art, in light of the teachings herein.

Some other nonbasic functionalized silicon compounds that are also useful in or as the nonbasic coating component of the duplex coating system of the invention are disclosed in the DS420 application and include: alkoxy-substituted branched silicones of intermediate size, having for example from about 10 to about 100 siloxy groups per molecule, for example, product KF-9908 supplied by Shin-Etsu Chemical Co., Ltd. (Tokyo, JP); polyacylate analogs of such branched polysiloxy compounds; polyfunctional silicon materials having more than two functional entities per unit; functionalized silicone compounds described in: Law, et al. U.S. Pat. No. 4,113,665 (Ameron), for example at column 2, lines 13 to 47 and column 3, line 17 to column 7, line 19; Socci, et al. U.S. Pat. No. 4,832,944 (Revlon), for example, at column 2, lines 21-51; Hollenberg, et al. U.S. Pat. No. 5,143,722, for example at column 2, line 43 to column 3, line 62; Hasegawa U.S. Pat. Nos. 5,368,639 and 5,458,681 (Miyoshi Kasei), for example at column 2, line 24 to column 2, line 48 of the '639 patent; Mitchnick, et al. U.S. Pat. No. 5,486,631 (Siltech and SunSmart), for example at column 2, line 49 to column 4, line 38; Mitchnick, et al U.S. Pat. No. 5,536,492 (Siltech and SunSmart); Horino, et al. U.S. Pat. No. 6,200,580 (Miyoshi Kasei) for example at column 3, lines 33-53 and column 6, line 55 to column 7, line 67; and Colton, et al. United States Patent Application 20020061407 (PPG) for example at paragraphs [0020]-[0022]. The specific passages cited, as well as the entire disclosures, of each of the patent publications identified in this paragraph are hereby incorporated herein by this specific reference thereto.

Some other suitable functionalized silicone compounds useful in the practice of the invention as or in the nonbasic coating component include fluorinated or alkyl fluorinated analogs of the silicone compounds described in the foregoing patents. Such useful fluorinated silicon compounds can have the desirable structural characteristics for fluorinated or alkyl fluorinated functionalized silicone compound starting materials that are described in parent U.S. patent application Ser. No. 10/791,424 which specific disclosures are hereby incorporated herein by this reference thereto.

Suitable Powder Materials. Powder materials suitable for use in the practice of the present invention include a wide range of inorganic pigments and organic pigments, pigment extenders and fillers and especially most if not all insoluble powder materials employed in the cosmetics arts. Suitable powder materials for treatment by the methods of the invention can have any appropriate size and/or size distribution, as will be apparent to those skilled in the art, including, for example employed can have a mean particle size of from about 0.01 (about 10 nm) to about 100 micron (also rendered as “μm” herein), preferably from about 0.01-20 μm. Powder materials having a mean particle size of from about 0.1 to about 10 μm are, without limitation, believed to be particularly useful. The powder materials may have any desired morphology such as approximately spherical, ovoid, apicular, laminar, rhomboid or other useful morphology as is known or will become known to those skilled in the art.

One useful class of powders to which the inventive treatments may be applied comprises color lakes. Color lakes, or “lakes” are usually regarded as a dye or other colorant supported on particles, or a powder, of an inert, insoluble, colorless, solid extender, typically a salt of a metal such as aluminum, barium, calcium, strontium or a salt of phosphotungstic or phosphomolybdic acids. One common useful substrate or extender is alumina.

Conventional treated or untreated lakes have several drawbacks which limit their applications. The can be difficult to handle, commercially available products typically being fine somewhat hydrophilic powders that readily adhere to containers, the skin and other ambient surfaces. In use color lakes may bleed that is to say, absorbed organic dye washes off the inorganic substrate in use. Also, even with lakes which exhibit small amounts of bleed at relatively neutral pH, such as from about pH 4 to pH 9, in higher or lower, strongly alkaline or acidic conditions color lakes may discharge their color into ambient media in a soluble form. Moreover, conventional color lakes may also be sensitive to attack and discoloration by light and chemical agents, for example reducing agents. Their hydrophilic surface character, as well as the motility of the dye or colorant component, can making effective coating difficult. Accordingly, color lakes can benefit from the coating treatments of this invention which, in most cases, can be expected to control one or more of these drawbacks and, in particular, may control, or eliminate, bleed, especially bleeding into aqueous dispersion media in which the coated color lake particles are suspended.

Any suitable color lake may be beneficially treated with the inventive duplex coating system and process, as will be apparent to those skilled in the art, including aluminum, barium, calcium, strontium and zirconium lakes of: FD&C Red No. 2, Red No. 4, Red No. 6, Red No. 7, Red 21, Red No. 27 and Red No. 40; FD&C Yellow No. 5 and No. 6; FD&C Blue No. 1 and No. 2; FD&C Green No. 3; FD&C Violet No. 1; C.I. Acid Yellow No. 36; C.I. Mordant Orange No. 1, Orange B, C.I. Vat Blue No. 1; C.I. Basic Violet No. 1; C.I. Basic Green No. 4; and C.I. Basic Blue No. 9.

Other suitable lakes or equivalent powders that may be coated by the invention will be apparent to those skilled in the art. Multiple lakes or other powders may be treated individually and then mixed into a final or intermediate product, or they may be mixed prior to treatment and treated conjointly.

Powder substrates treated pursuant to the invention can also include any suitable organic pigments, as known to those skilled in the art, for example pigments incorporating various aromatic dyes such as azo, indigoid, triphenylmethane, anthraquinone, hydroquinones and xanthine dyes, and other D&C and FD&C colors as well as the lakes of these colors, as are known in the art.

If desired, pearlescent pigments include titanated mica, fish scale white, bismuth oxychloride, titanated mica treated with iron oxide, mica titanium treated with Prussian blue, titanated mica treated with carbon black, titanated mica treated with carmine, and the like can be treated with the duplex coating system of the invention. Other powders as disclosed in the aforesaid applications may also be treated.

Proportions. In practicing the present invention, the proportion of the weight of the duplex coating system, comprising basic and nonbasic coating components and excluding the weight of any solvent, to the weight of powder to be treated will depend upon the nature of the substrate and desirably is sufficient to completely coat essentially every particle when thoroughly mixed with the dry powder substrate. A suitable proportion, based upon the weight of color lake, organic pigment or other powder or particulate material to be coated, is from about 0.1 to about 30 percent, preferably from about 2 to about 15 percent and more preferably from about 5 to about 10 percent.

Desirably, the relative proportions of basic to nonbasic coating component is from about 10:1 to 1:10, for example from about 2:1 to about 1:2. One useful range of proportions is from about 1.2:1 to about 1:1.2, for example about 1:1.

Coating Process. Any suitable process may be used for coating the cosmetic powders with the duplex coating agent. However, one useful hydrophobizing process comprises the following process elements:
a) thoroughly mixing the basic duplex coating component with the particulate powder material to be coated, preferably in a liquid dispersion medium;
b) filtering the resulting slurry to remove excess liquid and yield a paste;
c) mixing the nonbasic duplex coating component with the paste;
c) heating the paste to remove residual liquid components, cure the coating and yield a dry coated powder material; and
d) pulverizing the dried powder to the desired particle size.

A desirable objective is to conduct the process of the invention so as to coat each powder particle evenly and thoroughly with a uniform coating that essentially leaves no areas on the particle surface exposed to become sites of undesired reactions, for example, with end-product excipients or other ingredients.

Mixing element a) of the process can be effected in various ways, as will be understood by those skilled in the art. For example, employing an aqueous dispersion medium, a liquid basic or nonbasic coating component can be added to an aqueous slurry of the powder to be coated in the dispersion medium.

Alternatively, and preferably, the duplex coating components are separately dissolved in a suitable organic solvent for example isopar, especially isopar C and the solution is sprayed onto the powder and mixed well. Isopar is a partially neutralized mixture of isoparaffinic acids and isopar C comprises C7-C8 solvents.

Other suitable solvents for the duplex coating agent may be employed as known to those skilled in the art, for example different grades of isopar, such as isopar E or isopar G, isoheptane, isooctane, isononane, and petroleum distillates such as those available from Phillips Chemical under the trade names or trademarks Soltrol 130, Soltrol 150 and Soltrol 170. Any suitable proportion of solvent may be used, for example, from about 0.5:1 to about 10:1 by weight of the respective coating component, for example, a proportion of about 4:1 solvent to coating component.

As is well understood in the art, mixing should be continued until the mixture is well mixed, smooth and uniform.

If desired the basic and nonbasic coating components can be blended together and simultaneously applied to the powder to be coated. Any such blend desirably is freshly made and promptly applied to the powder.

Desirable that at least the nonbasic coating component should have fresh activity, being obtained from an unopened or only recently opened container, or stored under anhydrous conditions or otherwise protected from degradation of its active functional groups, or being recently synthesized, to ensure that it will be effective in the treatment process of this invention. Products such as the GE SF 1706 amine functional fluid which, as supplied, is a stock blend of a basic, amine-containing functionalized polysiloxane and a nonbasic polysiloxane functionalized with backbone alkoxy units, may be subject to attenuation of the activity of the nonbasic polysiloxane functional groups by the action of the amino congener and/or impurities, which loss of activity may accelerate after opening, owing to the action of moisture. Accordingly, it is desirable that the nonbasic coating component not be stored in intimate admixture with the basic coating component or other compounds that could compromise the availability of the functional groups in the coating process of the present invention.

Curing. As described above, after the particles are coated with the duplex coating system, desirably they are subjected to curing at an elevated temperature for a limited period of time. The combination of time and temperature desirably are effective to substantially complete useful chemical reactions that will yield a hydrophobic coating covalently bonded to the powder substrate without causing undesirable decomposition of the coating components or the powder substrate. Desirably also, curing is effected so as to drive off any solvent employed. The curing time may be such as yields a constant product weight, indicating that solvent loss is complete.

Some examples of suitable elevated temperatures are in the range of from about 50 to about 200° C., and of suitable times in the range of from about thirty minutes to about twenty-four hours. In one useful embodiment, the elevated temperature lies in the range of from about 70 to about 140° C. Another useful embodiment employs a temperature in the range of from about 90 to about 120° C. Useful time periods can be from about one to about six hours. In one embodiment of the invention, curing is effected for from about two to about four hours at a temperature in the range of from about 100 to about 120° C. Those skilled in the art will understand that suitable conditions can be varied and that the time period can usually be adjusted in an inverse relationship to the temperature employed.

Other suitable times and temperatures will be known to those of ordinary skill in the art, having regard to the materials employed, or can be determined without undue experimentation. Optionally drying may be conducted under vacuum.

If desired, the coated powder may be only partially cured at elevated temperature, allowing curing to be completed at ambient temperature. However little benefit is seen in such embodiments.

Another embodiment of the invention employs an additional curing or partial curing stage which is effected after application of the basic duplex coating component and before application of the nonbasic duplex coating component. Such a partial curing stage may comprise a short period, for example from about 5 to about 30 minutes at mildly elevated temperature, for example from about 50° C. to about 100° C., sufficient to ensure covalent bonding of the basic coating component to the powder substrate and to initiate solvent volatilization. Final curing is then effected after application of the nonbasic coating component, as described hereinabove.

If desired curing may be conducted under vacuum to facilitate solvent removal. Curing may be continued until the paste is dry, as may be determined by weight loss determination, if desired.

Pulverization of the dried powder can then be effected on the dried product to break up particle agglomerations or accretions and to obtain a desired particle size, optionally with separation and removal of undesired size fractions. Pulverization can be effected in conventional manner, for example using a mill, such as a jet mill, hammer mill, or the like, desirably without breaking particle substrates to expose untreated surfaces.

Coated powders. Coated powders according to the invention preferably comprise a thin, coherent homogenous film or layer being the residue of the duplex coating system, which film or layer is desirably tenaciously covalently bound to the particulate substrate. The coating is hydrophobic and preferably completely covers each particle, preventing egress of dyes or other colorants from the powder substrate and also preventing ingress of reactive chemical agents, aqueous media, wetting agents, excipients or other ambient materials in the environment of the coated powder to the substrate particle material beneath the coating.

While the invention is not intended to be limited by any theory, it may be contemplated that the coating on a powder particle comprises a crosslinked network of the residues of the nonbasic and basic coating components covalently bonded to the powder surface. Basic coating component residues may be coupled to both the powder substrate and nonbasic coating component residues through nitrogen or other multivalent basic group atoms, derived e.g. from amino groups, and through oxygen or other additional functional group atoms derived e.g. from alkoxy groups. Nonbasic coating component residues are coupled primarily to basic coating component residues, and secondarily to the powder substrate, through oxygen or other additional functional group atoms derived e.g. from alkoxy groups. Desirably, all or substantially all the nonbasic coating component reactive groups are fully reacted, and the nonbasic coating component residues completely cover the powder particle surface, whereby the powder particle surface is largely or entirely free of reactive groups. For example, the outer surface may consist essentially of polysiloxy chains fully substituted with methyl, ethyl or other alkyl groups.

While the invention is to be limited not by any particular theory, the molecular structure of the coated particles may be understood to comprise a web of cross-linked basic component residues, many of which residues are also covalently bound to the substrate. The bonds between neighboring residues and the substrate are largely, or entirely effected through oxygen atoms derived from one or more of the functional groups such as alkoxy groups R¹O—, R⁷O—, R⁸O—, RO— or R′O— in the basic component molecule. The resultant links between adjacent residues may be Si—O—Si links and the links between the residues and the particle substrate may be —Si—O—P groups where P is an atom in the substrate having an available valence, for example, in the case of an inorganic powder, a metal. Alternatively, in the case of an organic or organic-laden powder, such as a lake, P may be a carbon atom. As a further, though less probable or less frequent, alternative, the connecting moiety between the silicon atom and the carbon atom may be a peroxy —O—O— group, the additional oxygen atom being derived from an available OH— group in surface moisture on the powder or from an organic hydroxyl group.

The duplex coated color lakes or other powders of the present invention can be incorporated in a wide range of cosmetic formulations in proportions known to those skilled in the art, for example, depending upon the product, the cosmetic powder may comprise from about 0.1 to about 99 percent by weight of the end-product formulation, with lower proportions of from about 0.1 to 25 weight percent being preferred in liquids and creams, more preferably from about 1 to about 10 percent by weight.

The excellent hydrophobicity of the duplex coated powders of the invention render them particularly suitable for oil-in-water or water-in-oil emulsions such as creams and lotions, wherein the hydrophobically coated pigments have a strong affinity for the oil phase and do not tend to migrate undesirably to the aqueous phase.

There is no particular limit to the cosmetic product into which the coated powders of the invention may be formulated. Such products include skin care compositions skin packs, sunscreens, body lotions, body powder compositions, makeup, compositions including face powder, foundation, eye shadow, blush, lipstick, eye liner and eye brow and so on.

More than one duplex coated powder according to the present invention can be employed in a given cosmetic formulation. Where multiple such powders are employed they may be coated with the basic component either separately or together.

Some non-limiting examples, pursuant to the invention, of the preparation of duplex coated powders will now be described and compared with prior art treatments.

### COMPARATIVE EXAMPLE A

### FD & C Blue No. 1 Treated with Amine Functional Silicone Alone

95 g of dry powdered FD & C blue no. 1 aluminum lake from Sensient Cosmetic Technologies (LCW) (S. Plainfield, N.J.) are weighed into a processor. 5 g of an amine functional silicone fluid (GE Silicones SF1706) in a 20% wt/wt solution isopar are added dropwise to the color lake with mixing until homogenous. The processor is discharged and the resulting product is then cured for 3 hours at 105° C., or until there is no loss of weight. The product is an agglomerated powder which is lightly pulverized to yield a uniform, finely divided powdered product.

### COMPARATIVE EXAMPLE B

### Varying Proportions

Comparative Example A is repeated employing 1 g decrements of FD & C blue no. 1 aluminum lake down to 90 g, along with respective 1 g increments of amine functional silicone fluid up to 10 g, providing samples employing from 6 to 10% by weight of the sample of coating material.

### COMPARATIVE EXAMPLE C

### Other Color Lakes

Comparative Examples A and B are repeated employing FD & C yellow no. 5 aluminum lake or FD & C red no. 40 aluminum lake in place of FD & C blue no. 1 aluminum lake.

### COMPARATIVE EXAMPLE D

### Color Lake Treated with Trialkoxysilane Alone

Comparative Examples A-C are repeated employing similar quantities of triethoxy caprylylsilane Sensient Cosmetic Technologies (LCW) (S. Plainfield, N.J.) in a 20% wt/wt solution in isopar in place of the amino functional silicone fluid.

### EXAMPLE 1

### Duplex Coating Treatment of FD & C Blue No. 1 Aluminum Lake

95 g of dry powdered FD & C blue no. 1 aluminum lake from Degussa Corporation (Ridgefield Park, N.J.) are weighed into a processor. 2.5 g of an amine functional silicone fluid (GE Silicones SF1706) in a 20% wt/wt solution in isopar are added dropwise to the color lake with mixing until homogenous. After the amino functional silicone solution has been completely added, 2.5 g of triethoxy caprylylsilane in a 20% wt/wt solution is added dropwise to the amino functional silicone coated color lake powder, before curing, and is mixed to homogeneity. The processor is discharged and the resulting product is then cured for 3 hours at 105° C., or until there is no loss of weight. The cured product, an agglomerated powder, is lightly pulverized to yield a uniform, finely divided powder of good quality, homogenous appearance and good feel.

### EXAMPLE 2

### Varying Proportions

Example 1 is repeated employing 1 g decrements of FD & C blue no. 1 aluminum lake down to 90 g, along with respective 0.5 g increments of amine functional silicone fluid up to 5 g and 0.5 g increments of triethoxy caprylylsilane up to 5 g, providing samples employing from 6 to 10% by weight of the sample having a duplex coating treatment. Comparable powders are produced.

### EXAMPLE 3

### Duplex Coating Treatment of FD & C Red No. 40 Aluminum Lake

Examples 1 and 2 are repeated employing FD & C red no. 40 aluminum lake in place of FD & C blue no. 1 aluminum lake. Comparable powders are produced.

### EXAMPLE 4

### Duplex Coating Treatment of FD & C Yellow No. 5 Aluminum Lake

Examples 1 and 2 are repeated employing FD & C Yellow no. 5 aluminum lake in place of FD & C blue no. 1 aluminum lake. Comparable powders are produced.

Hydrophobicity Test. 1 g samples of each of the coated powder products of Comparative Examples A-D and Examples 1-4 are separately shaken with a 50 g aqueous aliquot pH adjusted to 7. The ability of the coated powder to float on the aqueous aliquot is indicative of the quality of the hydrophobic coating.

Bleed Test. Bleed into the aqueous phase of the hydrophobicity aliquot is determined by qualitative visual assessment of coloration. If desired, a colorimeter can be used to quantify the color intensity, though such data are not described here.

Results. Some of the results obtainable with products such as those described in the foregoing comparative Examples A-D and invention Examples 1-4 are illustrated in Tablel, below.

TABLE 1
Comparative Bleed Results of Treated Lakes

| | Water Solubility | Hydrophobicity | Bleed - 1 day | Bleed - 1 week |
|---|---|---|---|---|
| | (uncoated) | (coated) | (coated) | (coated) |
| Comparative Ex. A-B | partially | floats | some | NI |
| FD&C Blue No 1 Al Lake | | some settling | | |
| Comparative Ex. C | slightly | floats | some | NI |
| FD&C Red no 40 Al Lake | | some settling | | |
| Comparative Ex. C | slightly | floats | some | NI |
| FD&C Yellow No 5 Al Lake | | some settling | | |
| Comparative Ex. D | partially/slightly | floats | some | NI |
| Triethoxysilane | | some settling | | |
| Ex. 1-2 Duplex Coating 5% | partial | floats | little or none | little or none |
| FD&C Blue No 1 Al Lake | | some settling | | |
| Ex. 1-2 Duplex Coating 6% + | partial | floats | little or none | little or none |
| FD&C Blue No 1 Al Lake | | | | |
| Ex. 3 Duplex Coating 5% | slightly | floats | little or none | little or none |
| FD&C Red No 40 Al Lake | | some settling | | |
| Ex. 3 Duplex Coating 6% + | slightly | floats | little or none | little or none |
| FD&C Red No 40 Al Lake | | | | |
| Ex. 4 Duplex Coating 5% | slightly | floats | little or none | little or none |
| FD&C Yellow No 5 Al Lake | | | | |
| Ex. 4 Duplex Coating 6% + | slightly | floats | little or none | little or none |
| FD&C Yellow No 5 Al Lake | | | | |

NI = not interesting

As shown in Table 1, blue 1 lake is partially soluble in water while red 40 lake and yellow 5 lake are slightly soluble in water. When treated with similar proportions of the individual components, namely amino functional silicone, Comparative Examples A-C or triethoxy silane, Comparative Example D, the treated powders may all bleed within the first day. The treated powders largely float on water but show minor settling demonstrating fair but not ideal hydrophobicity.

After coating and curing with the duplex treatment of the invention, pursuant to Examples 1-4, the pigment not only does the treated powder float well on water demonstrating hydrophobicity, but the bleed into water is almost non-existent.

Samples of the invention treated powder kept in water for a full week show almost no bleed.

The treated FD&C yellow no. 5 lake of Example 4 is good at 5%, with virtually no settling, and the performance is still better as the amount of coating increases. For FD&C red no. 40 and FD&C blue no. 1, similar improvement may be occur at a coating proportion of about 6%. Exceptionally good results can be obtained with all three lakes employing higher proportions of coating ingredients, for example up to about 10% of the coated powder by weight.

The good and excellent hydrophobicity obtainable with the inventive treatments is attractive for cosmetic formulations especially where the powders are dispersed in liquid media, while the avoidance of bleed solves a long-standing problem with the use of many color lakes. Thus color lake powders treated pursuant to the invention are uniquely appealing to cosmetics and other formulators. Furthermore, some embodiments of inventive treated color lakes may have enhanced handling properties, good pourability, and exhibit less propensity to adhere to container surfaces, clothing, skin or other ambient surfaces.

As may be understood from the foregoing disclosure, the present invention provides a novel cosmetic powder treatment process and novel hydrophobically treated cosmetic powders. Preferred embodiments of the invention can be employed to produce an effective hydrophobic coating on a wide variety of useful and commercially significant cosmetic powders. Excellent or superior water repellency, stability with good shelf life and no outgassing, smooth feel and good adhesion to the skin are obtainable in cosmetic formulations in which preferred embodiments of the invention are employed.

In another broad aspect, the invention provides a solution to problems of producing a high quality hydrophobic silicone coating on powders, especially, but not exclusively, powders that are difficult to coat, and/or powders that are to be dispersed in a liquid or solid phase dispersion medium, by providing a two-stage treatment process employing a duplex coating system comprising a reactive component and a supplement component. In a first stage of the process, the reactive coating component is applied to the powder to be treated. The reactive coating component desirably is selected to react with and bond to the powder surface. In a second stage, the supplemental coating component providing is applied to the powder coated with the reactive coating component. The supplemental coating component is selected to yield a desired, optionally hydrophobic, outer molecular layer after curing of the coated powder. The coated powder is then cured to yield a product having a desired outer molecular layer, e.g. hydrophobic, covalently bonded to the powder substrate. The reactive coating component desirably has functional groups such as aminopolysiloxy or organometallate groups that will efficiently covalently couple to the surface of the powder substrate, and may usefully be any similar to the basic coating component described hereinabove. The supplemental coating component can be selected to efficiently covalently couple to the powder coated with the reactive coating component, and to enable provision of the desired outer molecular layer. The outer molecular layer may optionally be a hydrophobic silicone layer, a hybrid comprising a cross-linked network of siloxy and metalloxy units, with or without alkyl substituents, or other desired outer layer, as will be understood by those skilled in the art in light of the teaching herein. The supplemental coating component may be similar to the nonbasic coating component described hereinabove.

Preferably the outer layer of the cured coated particulate powder material is relatively free of reactive groups or groups that are antipathetic to hydrophobicity, particularly, but not exclusively, hydrophilic groups. To this end, the chemical character of the supplemental coating agent can be selected to have reactive groups which will become substantially or completely reacted during in the treatment process to leave no or little reactive residue. For example the supplemental a

Desirably the outer molecular layer comprises a coherent complete coating on the powder particle substrate, and desirably also, substantially all the powder particles in the product are so coated. Particles that are not satisfactorily coated, should any exist, can, if desired, be removed, for example by agglomeration of the unsatisfactorily coated particles and separation of the agglomerations from the effectively coated powders, in known manner.

The additional complication and expense of carrying out a two-stage process can be useful in leading to the provision of a coated powder having an external molecular layer of a desired chemical character covalently bonded to a substrate powder in cases where the respective chemistries of the second stage coating component and the powder do not in and of themselves permit a desired external coating to be obtained.

Although the invention has been described primarily in terms of a duplex coating system having two coating components, it will be understood that coating systems having three or more components can also serve the objectives of the invention and fall within its scope.

The present invention includes the coated powder products of the treatment processes and methods described herein as well as cosmetic formulations, paints and other coatings, plastics, rubbers or other end-product formulations in which the inventive coated powders may be employed. It will be understood that treated powders produced by the methods of the invention and providing one or more benefits of the invention may have a chemical structure such as is described or suggested herein or may have a modified or alternative chemical structure.

### INDUSTRIAL APPLICABILITY

While the present invention has primarily been described as it applies to novel hydrophobic cosmetics powders and to cosmetic formulations employing such cosmetic powders, it will be understood by those skilled in the relevant art or arts that the invention may be beneficially applied in other industries, for example in the paint and coatings industries or the plastics industry, where it is desired to disperse powders, including pigment powders, in liquid or solid phase media.

In addition to the foregoing cosmetics and other applications, the inventive treated color lakes described hereinabove, especially treated FD&C lakes, are useful to food processors, e.g. for formulation of non-aqueous or low moisture content products including hard fat coatings, frosting sugars, icings and fondant coatings, cake and doughnut mixes, variegating sauces, dry beverage and dessert powders, snack foods, pet foods, and various tablet coatings for the confection and pharmaceutical industries. In addition, FD&C, and, in certain cases, D&C, treated color lakes can be used not only in cosmetics e.g. in lotions, creams, lipsticks, powders and soaps but also in packaging materials for the food and pharmaceutical industries for example for inks, films, coatings and can liners. The treated FD&C, D&C, and noncertified color lakes of embodiments of the invention can also find application in other industries for example in textile dyeing and in other pigment applications such as in lithographic and printing inks, in artist colors, and in crayons.

Disclosures Incorporated. The entire disclosure of each and every United States patent and patent application, each foreign and international patent publication, of each other publication and of each unpublished patent application that is referenced in this specification or elsewhere in this patent application, is hereby incorporated herein, in its entirety, by the respective specific reference that has been made thereto.

While illustrative embodiments of the invention have been described above, it is, of course, understood that various modifications will be apparent to those of ordinary skill in the art. Many such modifications are contemplated as being within the spirit and scope of the invention.

1. A process for treating a powder, optionally a cosmetic powder, to render the powder hydrophobic, the process comprising treating the powder with a duplex coating system comprising an effective amount of a basic coating component and an effective amount of a nonbasic coating component, by applying the basic coating component to the powder so as to substantially coat the powder with the basic coating component, then applying the nonbasic coating component to the powder coated with the basic coating component, with mixing and curing the resultant powder coated with the basic and nonbasic coating components.

2. A process according to claim 1 wherein the basic and the nonbasic components of the duplex coating system each comprises a functionalized organosilicon compound, the basic component functionalized organosilicon compound containing reactive basic groups and, optionally, a second organosilicon compound lacking reactive basic groups.

3. A process according to claim 1 wherein the nonbasic coating component comprises a functionalized silane having from one to four silicon atoms.

4. A process according to claim 2 wherein the basic coating component comprises a polysiloxane substituted with multiple amino or other basic nitrogen groups and with multiple methoxy or ethoxy groups.

5. A process according to claim 2 wherein the nonbasic coating component comprises a trimethoxy- or triethoxy-alkylsilane having a single silicon atom.

6. A process according to claim 2 wherein the powder or powders to be treated is selected from the group consisting of cosmetic powders, color lakes, organic pigment powders, hard-to-coat powders, powders that have hydrophilic outer surfaces, pearlescent pigments, mica-based pigments, hard-to-coat pigments, pigment extenders, fillers and mixtures of two or more of the foregoing powders.

7. A process according to claim 2 wherein the powder comprises a color lake, optionally a color lake selected from the group consisting of aluminum, barium, calcium, strontium and zirconium lakes of: FD&C Red No. 2, Red No. 4, Red No. 6, Red No. 7, Red 21, Red No. 27 and Red No. 40; FD&C Yellow No. 5 and No. 6; FD&C Blue No. 1 and No. 2; FD&C Green No. 3; FD&C Violet No. 1; C.I. Acid Yellow No. 36; C.I. Mordant Orange No. 1, Orange B, C.I. Vat Blue No. 1; C.I. Basic Violet No. 1; C.I. Basic Green No. 4; and C.I. Basic Blue No. 9.

8. A process according to claim 7 comprising employing an amount of from about 2 to about 30 percent by weight, based on the weight of the powder to be coated, of the duplex coating system wherein, optionally, the proportion of basic to nonbasic coating component is from about 10:1 to about 1:10.

9. A process according to claim 8 comprising completely coating the powder with the basic coating component and then coating the powder with the nonbasic coating component.

10. A process according to claim 1 wherein the nonbasic coating component is applied to the powder after application of the basic coating component has been completed, in a two-stage treatment.

11. A process according to claim 2 wherein the basic component functionalized organosilicon compound comprises a dialkylpolysiloxane having basic groups substituted in a repeating backbone unit and optionally also having electronegative functional groups substituted in the backbone.

12. A process according to claim 2 comprising mixing the basic coating component with the nonbasic coating component and coating the powder with the fresh mixture.

13. A process according to claim 2 comprising mixing the nonbasic coating component with the powder to coat the powder and then mixing the basic coating component with the powder coated with the nonbasic coating component.

14. A process according to claim 2 wherein the powder is insoluble in aqueous and organic media and the basic component has no silicon-hydrogen bonds.

15. A process according to claim 2 wherein the basic coating component further comprises a nonbasic organosilicon compound, optionally being a dialkyl polysiloxane having alkoxy groups substituted in its backbone.

16. A process according to claim 2 wherein the basic coating component is selected from the group consisting of: a compound complying with Formula (1) herein; a compound complying with Formula (2) herein; a compound complying with Formula (1) herein together with a similar compound lacking the basic group; a compound complying with Formula (2) herein together with a similar compound lacking the basic group; and a compound complying with Formula (2) together with a compound complying with Formula (3) herein.

17. A process according to claim 2 comprising the following process elements:
a) thoroughly mixing the basic coating component with the powder to be treated, optionally in a liquid dispersion medium;
b) thoroughly mixing the nonbasic coating component with the powder coated with basic coating component;
c) filtering the resulting slurry to remove excess liquid and yield a paste;
d) heating the paste to remove residual liquid components, cure the coating and yield a dry coated powder material; and
d) pulverizing the dried powder to a desired particle size.

18. A coated powder comprising insoluble powder particles coated with a film comprising a cross-linked web of the residues of a basic coating component and a nonbasic coating component.

19. A coated powder produced by the method of claim 1.

20. A cosmetic composition comprising from about 0.1 to about 99 percent by weight of a coated powder produced by the method of claim 1.

21. A duplex coating system comprising an effective proportion of:
a) a basic coating component comprising a polysiloxane having backbone-substituted basic groups; and
b) a functionalized silane having not more than four silicon atoms.

22. A two-stage process for treating powders for dispersal in a liquid- or solid-phase dispersion medium to provide a durable external coating on the powder particles, the process comprising:
a) employing a duplex coating system comprising a reactive coating component selected to react with and bond to the powder surface and a supplemental coating component selected to yield a desired, optionally hydrophobic, outer molecular layer after curing of the coated powder;
b) in a first stage of the process applying the reactive coating component to the powder to be treated;
c) in a second stage, applying the supplemental coating component to the powder coated with the reactive coating component; and
d) curing the coated powder to yield a product having a desired outer molecular layer, optionally hydrophobic, covalently bonded to the powder substrate.

23. A process according to claim 22 wherein the reactive coating component has functional groups optionally aminopolysiloxy or organometallate groups and the supplemental coating component can provide a hydrophobic silicone outer layer.
